(19) United States
(12) Patent Application Publication
(10) Pub. No.: US 2025/0170051 A1
(43) Pub. Date: May 29, 2025

(54) Personal Care Products

(71) Applicant: Colgate-Palmolive Company, New York, NY (US)

(72) Inventors: Savitha RAJAGOPAL, Chandler, AZ (US); Ewelina LESNIAK, North Brunswick, NJ (US); Alexandra Draganescu D'ARCANGELIS, Randolph, NJ (US)

(73) Assignee: Colgate-Palmolive Company, New York, NY (US)

(21) Appl. No.: 18/962,927

(22) Filed: Nov. 27, 2024

Related U.S. Application Data
(60) Provisional application No. 63/603,902, filed on Nov. 29, 2023.

(51) Int. Cl. A61K 8/9789; A61K 8/02; A61K 8/365; A61K 8/67; A61Q 19/00

(52) U.S. Cl. CPC A61K 8/9789; A61K 8/0208; A61K 8/365; A61K 8/673; A61K 8/675; A61Q 19/00

(57) ABSTRACT

Personal care products and compositions for exfoliating and brightening skin are provided herein. In accordance with an aspect, provided is a personal care product comprising a personal care composition comprising a hydroxy acid, prickly pear extract, and vitamin B. According to another aspect, the personal care formulation comprises alpha hydroxy acid and/or beta hydroxy acid.

### CROSS-REFERENCE TO RELATED APPLICATION

This application claims the benefit of priority from U.S. Provisional Application No. 63/603,902, filed Nov. 29, 2023, titled Personal Care Products, the contents of which are incorporated herein by reference in its entirety for all purposes.

### BACKGROUND

Skincare products have many benefits including recuperating a youthful and glowing appearance. While skin could exfoliate dead skin cells naturally, the exfoliation process slows and may stop altogether as a result of various factors such as the aging process, sun damage, or exposure to environmental pollutants. In such cases, skin appears dull, dry and flaky. By gently exfoliating the buildup of dead skin, the skin's beauty and health are restored.

Many personal care products currently available to consumers are directed primarily to improving the health and/or appearance of skin. For example, there are a variety of topical skin care products available that are directed to delaying, minimizing, or even eliminating skin dryness, skin wrinkling, and other histological changes commonly associated with the aging of skin or environmental damage to human skin.

### BRIEF SUMMARY

Aspects of the invention are directed to personal care products and compositions, particularly, to a personal care product for improving skin health through the application of a personal care composition impregnated on a pad. The personal care compositions of the invention exfoliate, improve skin texture, and brighten the skin by lifting away dead skin cells and encouraging cell turnover.

In accordance with one aspect of the invention, provided is a personal care composition comprising a plurality of hydroxy acids, Opuntia ficus-indica (prickly pear) extract, and vitamin B. In some embodiments, the plurality of hydroxy acids may comprise an alpha hydroxy acid and/or a beta hydroxy acid. The alpha hydroxy acid may be selected from glycolic acid, citric acid, lactic acid, malic acid, tartaric acid, phytic acid, hydroxycaprylic acid, hydroxycapric acid, and a combination of two or more thereof. The beta hydroxy acid may be selected from salicylic acid, sodium salicylate, beta hydroxybutanoic acid, tropic acid, trethocanic acid, and a combination of two or more thereof.

In some embodiments, the plurality of hydroxy acids may comprise a polyhydroxy benzoic acid, a polyhydroxy benzoic ester, or a combination thereof. The polyhydroxy benzoic acid compound may be selected from 2-hydroxy benzoic acid, 3-hydroxy benzoic acid, 4-hydroxy benzoic acid, 2, 3-dihydroxy benzoic acid, 2, 4-dihydroxy benzoic acid, 2, 5-dihydroxy benzoic acid, 2, 6-dihydroxy benzoic acid, 3, 4-dihydroxy benzoic acid, 3, 5-dihydroxy benzoic acid, 2,3, 4-trihydroxy benzoic acid, 2,4, 6-trihydroxy benzoic acid, 3,4, 5-trihydroxy benzoic acid, an ester thereof, and a combination of two or more thereof.

A list of non-limiting, example embodiments in accordance with certain aspects of the invention is provided below:

In accordance with an embodiment 1, provided is a personal care product comprising: a pad containing a personal care composition, the personal care composition comprising: from about 2 to about 23 wt. % of a plurality of hydroxy acids; from about 0.01 to about 7 wt. % of prickly pear; and from about 1 to about 12 wt. % of vitamin B.

In accordance with an embodiment 2, provided is the personal care product according to embodiment 1, wherein the plurality of hydroxy acids comprise an alpha hydroxy acid and a beta hydroxy acid.

In accordance with an embodiment 3, provided is the personal care product according to embodiment 1 or embodiment 2, wherein the plurality of hydroxy acids comprise an alpha hydroxy acid selected from glycolic acid, citric acid, lactic acid, mandelic acid, malic acid, tartaric acid, phytic acid, hydroxycaprylic acid, hydroxycapric acid, and a combination of two or more thereof.

In accordance with an embodiment 4, provided is the personal care product according to embodiment 2 or embodiment 3, wherein the alpha hydroxy acid comprises mandelic acid, lactic acid, malic acid, gluconic acid, tartaric acid, phytic acid, or a combination of two or more thereof.

In accordance with an embodiment 5, provided is the personal care product according to one of embodiment 2 to embodiment 4, wherein the alpha hydroxy acid is present in an amount from 1 to about 16 wt. %, based on the total weight of the personal care composition.

In accordance with an embodiment 6, provided is the personal care product according to one of embodiment 1 to embodiment 5, wherein the plurality of hydroxy acids comprise a beta hydroxy acid selected from salicylic acid, sodium salicylate, beta hydroxybutanoic acid, tropic acid, trethocanic acid, and a combination of two or more thereof.

In accordance with an embodiment 7, provided is the personal care product according to one of embodiment 2 to embodiment 6, wherein the beta hydroxy acid comprises salicylic acid, sodium salicylate, or a combination thereof.

In accordance with an embodiment 8, provided is the personal care product according to one of embodiment 2 to embodiment 7, wherein the beta hydroxy acid is present in an amount from about 0.5 to about 10 wt. %, based on the total weight of the personal care composition.

In accordance with an embodiment 9, provided is the personal care product according to any foregoing embodiment, wherein the plurality of hydroxy acids comprise a polyhydroxy benzoic acid, a polyhydroxy benzoic ester, a hydrate thereof, or a combination thereof.

In accordance with an embodiment 10, provided is the personal care product according to embodiment 9, wherein the polyhydroxy benzoic acid compound is selected from 2-hydroxy benzoic acid, 3-hydroxy benzoic acid, 4-hydroxy benzoic acid, 2, 3-dihydroxy benzoic acid, 2, 4-dihydroxy benzoic acid, 2, 5-dihydroxy benzoic acid, 2, 6-dihydroxy benzoic acid, 3, 4-dihydroxy benzoic acid, 3, 5-dihydroxy benzoic acid, 2,3, 4-trihydroxy benzoic acid, 2,4, 6-trihydroxy benzoic acid, 3,4, 5-trihydroxy benzoic acid, an ester thereof, and a combination of two or more thereof.

In accordance with an embodiment 11, provided is the personal care product according to any foregoing embodiment further comprising a soothing agent.

In accordance with an embodiment 12, provided is the personal care product according to embodiment 11, wherein the soothing agent is present in an amount from about 0.01 to about 7 wt. %, based on the total weight of the personal care composition.

In accordance with an embodiment 13, provided is the personal care product according embodiment 11 or embodiment 12, wherein the soothing agent comprises Centella asiatica extract, Polygonum cuspidatum root extract, Scutellaria baicalensis root extract, Camellia sinensis leaf extract, Glycyrrhiza glabra (licorice) root extract, Chamomilla recutita (matricaria) flower extract, Rosmarinus officinalis (rosemary) leaf extract, tea (Camellia sinensis) leaf extract, Poria cocos extract, Phragmites karka extract, lidocaine, chia seed extract, Rosa centifolia flower extract, aloe vera, allantoin, D-panthenol, turmeric, avocado oil, and lichen extract, or a combination of two or more thereof.

In accordance with an embodiment 14, provided is the personal care product according to any foregoing embodiment further comprising a humectant, optionally, in an amount from about 1 to about 20 wt. %, based on the total weight of the personal care composition.

In accordance with an embodiment 15, provided is the personal care product according embodiment 14, wherein the humectant is selected from glycerin, butylene glycol, 1,3-propanediol, polyglutamic acid, saccharide isomerate, and a combination of two or more thereof.

In accordance with an embodiment 16, provided is the personal care product according to any foregoing embodiment, wherein the vitamin B is selected from vitamin B1, vitamin B2, vitamin B3, vitamin B5, vitamin B6, vitamin B12, a derivative thereof, and a combination of two or more thereof.

In accordance with an embodiment 17, provided is the personal care product according to any foregoing embodiment, wherein the vitamin B is selected from thiamine, riboflavin, niacinamide, pantothenic acid, and a combination of two or more thereof.

In accordance with an embodiment 18, provided is the personal care product according to any foregoing embodiment, wherein the vitamin B comprises niacinamide and/or a derivative thereof.

In accordance with an embodiment 19, provided is the personal care product according to any foregoing embodiment, wherein the plurality of hydroxy acids comprises two or more alpha hydroxy acids selected from lactic acid, glycolic acid, mandelic acid, gluconic acid, and/or salts thereof.

In accordance with an embodiment 20, provided is the personal care product according to any foregoing embodiment, wherein the plurality of hydroxy acids consists of one or more alpha hydroxy acid selected from lactic acid, glycolic acid, mandelic acid, gluconic acid, a salt thereof, and a combination of two or more thereof.

In accordance with an embodiment 21, provided is a personal care product comprising: a pad; and a personal care composition according to any foregoing embodiment, wherein the personal care composition is contained in the pad.

In accordance with an embodiment 22, provided is the personal care product according to embodiment 21, wherein the personal care composition is impregnated in the pad.

In accordance with an embodiment 23, provided is the personal care product according to embodiment 21 or embodiment 22, wherein the pad is formed of natural fibers.

In accordance with an embodiment 24, provided is the personal care product according to embodiment 21, wherein the pad comprises woven fibers.

In accordance with an embodiment 25, provided is the personal care product according to embodiment 21, wherein the pad comprises a gel phase.

### BRIEF DESCRIPTION OF THE DRAWINGS

Implementation of the present technology will now be described, by way of example only, with reference to the attached figures, wherein:

FIG. 1 is a graph showing the effect of a non-limiting, example composition on IL-la cytokine expression in accordance with aspects of the invention;

FIG. 2 is a graph showing the effect of a non-limiting, example composition on ELOVL4 gene expression in accordance with aspects of the invention;

FIG. 3 is a graph showing the effect of a non-limiting, example composition on KRT 5 gene expression in accordance with aspects of the invention;

FIG. 4 is a graph showing the effect of a non-limiting, example composition on KLK7 gene expression in accordance with aspects of the invention; and

FIG. 5 is a graph showing the effect of a non-limiting, example composition on filaggrin protein expression in accordance with aspects of the invention.

It should be understood that the various aspects are not limited to the arrangements and instrumentality shown in the drawings.

### DETAILED DESCRIPTION

It is to be understood that the foregoing detailed description of the invention and embodiments, are exemplary and explanatory only and are not restrictive in any way of the claimed invention. As used herein, the use of the plural tense includes the singular tense unless specifically stated otherwise. Similarly, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc. The term “about” when referring to a number means any number within a range of 10% of the number. For example, the phrase “about 2.0 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

All components and elements positively set forth in this disclosure can be negatively excluded from the claims. In other words, the personal care composition of the instant disclosure can be free or essentially free of all components and elements positively recited throughout the instant disclosure. In some instances, the personal care composition of the present invention may be substantially free of non-incidental amounts of the ingredient(s) or compound(s) described herein. A non-incidental amount of an ingredient or compound is the amount of that ingredient or compound that is added into the personal care composition by itself. For example, a personal care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the personal care composition includes both components (or the composition includes more than two components that overlap), an overlapping compound does not represent more than one component. For example, certain compounds (e.g., ethanol) may be characterized as both a penetration agent and a carrier. If a particular personal care composition includes both a penetration agent and a carrier, ethanol will serve only as either a penetration agent or a carrier—not both.

As used herein, reference to a compound comprising several isomers or stereoisomers includes all the isomeric or stereoisomeric forms of that compound. For example when reference is made to lactic acid, it can refer to l-(+)-lactic acid or d-(−)-lactic acid, or to a mixture thereof. Unless stated otherwise, all percentages of the personal care composition ingredients, given in this specification, are by weight based on the total personal care composition weight of 100%.

As used herein, reference to an acid, may include all chemical salts and/or esters of that acid. For example when reference is made to lactic acid, it can refer to a lactate salt or to a lactate ester. Some of the personal care composition of the invention may comprise ingredients which have several properties, for instance some of the anti-inflammatory ingredients may have soothing properties and/or wound healing properties as well as antipollution properties and antioxidation properties.

As used herein, the term “exfoliating” refers to a cosmetic technique which improves the skin appearance by removing or facilitating the removal of dead skin cells which accumulate on the topmost layer of skin and/or stratum corneum enabling the new layer of skin cells to come to the surface and grow.

The personal care composition of the invention may be applied on the skin of the human subject. In general, the personal care composition of the invention may be applied to the face, the back of the hands, the knees, the heels or on the skin of any other parts of the human subject. In some preferable embodiments, the personal care composition of the invention may be applied to the face.

As used herein, the term “topical application” refers to applying or spreading the personal care composition of the invention on the surface of the skin of the human subject.

Aspects of the invention are directed to personal care products comprising a personal care composition formulated to improve the appearance of skin and/or skin health. In one embodiment, the personal care product is a personal care composition applied to a pad. The personal care products disclosed herein may, in some embodiments, disclose dead cells and may have a watery, gel-like consistency. Chemical peels may comprise fruit enzymes, fruit acids, and/or metal complexes. The inventors discovered that certain combinations in particular amounts and ratios provide enhanced benefits to the skin when applied to the skin via a pad such as an exfoliating pad.

In accordance with one aspect of the invention, provided is a personal care product comprising a pad with a personal care composition comprising a plurality of hydroxy acids, Opuntia ficus-indica (prickly pear) extract, and vitamin B. According to another aspect of the invention, provided is a personal care product comprising a pad comprising a hydroxy acid, p Opuntia ficus-indica (prickly pear) extract, and vitamin B; wherein the personal care composition has a weight ratio of hydroxy acid to prickly pear that is about 1:2 to about 250:1, and wherein the personal care composition has a weight ratio of hydroxy acid to vitamin B that is about 1:3 to about 25:1. In one embodiment, the personal care composition comprises at least one hydroxy acid that is selected from alpha hydroxy acid or beta hydroxy acid.

The personal care composition typically comprises a plurality of hydroxy acids and/or salts thereof. The plurality of hydroxy acids and/or salts thereof may be present in the personal care composition in an amount, preferably, from about 2 to about 23 wt. %, about 2 to about 18 wt. %, about 2 to about 13 wt. %, about 2 to about 11 wt. %, about 2 to about 9 wt. %, about 2 to about 7 wt. %, about 2 to about 5 wt. %; from about 3 to about 23 wt. %, about 3 to about 18 wt. %, about 3 to about 13 wt. %, about 3 to about 11 wt. %, about 3 to about 9 wt. %, about 3 to about 7 wt. %, about 3 to about 5 wt. %; from about 4 to about 23 wt. %, about 4 to about 18 wt. %, about 4 to about 13 wt. %, about 4 to about 11 wt. %, about 4 to about 9 wt. %, about 4 to about 7 wt. %; from about 5 to about 23 wt. %, about 5 to about 18 wt. %, about 5 to about 13 wt. %, about 5 to about 11 wt. %, about 5 to about 9 wt. %; about 7 to about 23 wt. %, about 7 to about 18 wt. %, about 7 to about 13 wt. %, about 7 to about 11 wt. %; about 9 to about 23 wt. %, about 9 to about 18 wt. %, about 9 to about 13 wt. %; from about 11 to about 23 wt. %, about 11 to about 18 wt. %, about 11 to about 14 wt. %, from about 13 to about 23 wt. %, about 13 to about 18 wt. %, about 13 to about 16 wt. %; from about 15 to about 23 wt. %, about 15 to about 18 wt. %, or any range or subrange thereof, based on the total weigh of the personal care composition.

The plurality of hydroxy acids and/or salts thereof typically comprises one or more alpha hydroxy acid(s), e.g., present in an amount from about 2 to about 20 wt. %, based on the total weight of the personal care composition. For instance, the alpha hydroxy acid(s) may be present in the personal care composition in an amount from about 2 to about 18 wt. %, about 2 to about 16 wt. %, about 2 to about 14 wt. %, about 2 to about 12 wt. %, about 2 to about 10 wt. %, about 2 to about 8 wt. %, about 2 to about 6 wt. %, about 2 to about 4 wt. %; from about 5 to about 20 wt. %, about 5 to about 18 wt. %, about 5 to about 16 wt. %, about 5 to about 14 wt. %, about 5 to about 12 wt. %, about 5 to about 10 wt. %, about 5 to about 8 wt. %; from about 8 to about 20 wt. %, about 8 to about 18 wt. %, about 8 to about 16 wt. %, about 8 to about 14 wt. %, about 8 to about 12 wt. %, about 8 to about 10 wt. %; from about 10 to about 20 wt. %, about 10 to about 18 wt. %, about 10 to about 16 wt. %, about 10 to about 14 wt. %, about 10 to about 12 wt. %; from about 12 to about 20 wt. %, about 12 to about 18 wt. %, about 12 to about 16 wt. %, about 12 to about 14 wt. %; from about 14 to about 20 wt. %, about 14 to about 18 wt. %, about 14 to about 16 wt. %; from about 16 to about 20 wt. %, about 16 to about 18 wt. %, including any range or subrange thereof, based on the total weight of the personal care composition. In at least one instance, the personal care composition comprises polyhydroxy acid(s) from about 10 to about 15 wt. %, based on the total weight of the personal care composition.

The personal care composition may include one or more alpha hydroxy acid(s) and/or salt(s) thereof. For instance, the personal care composition may include one or more alpha hydroxy acid(s) and/or salt(s) thereof, such as those selected from C₃ to C₇ alpha-hydroxy acid or C₄ to C₆ alpha-hydroxy acid. Examples of alpha hydroxy acid and/or salts thereof include glycolic acid, citric acid, lactic acid, malic acid, mandelic acid, ascorbic acid, alpha-hydroxybutyric acid, alpha-hydroxyisobutyric acid, alpha-hydroxyisocaproic acid, atrrolactic acid, alpha-hydroxyisovaleric acid, ethyl pyruvate, galacturonic acid, glucopehtonic acid, glucopheptono 1,4-lactone, gluconic acid, glucuronic acid, glucurronolactone, glycolic acid, isopropyl pyruvate, methyl pyruvate, mucic acid, pyruvia acid, saccharic acid, saccaric acid 1,4-lactone, tartaric acid, tartronic acid, and salts thereof. In some embodiments, the alpha-hydroxy acid may be in the form of a salt. The salt of the alpha-hydroxy acid is preferably a sodium salt or a potassium salt. In at least one embodiment, the salt is a sodium salt (i.e., the cation associated with the salt is a sodium).

In certain embodiments, the one or more alpha hydroxy acid includes, but is not limited to, mandelic acid, glycolic acid, citric acid, lactic acid, malic acid, tartaric acid, phytic acid, hydroxycaprylic acid, hydroxycapric acid, glutaric acid, gluconic acid, a salt thereof, or a combination of two or more thereof. For example, the alpha hydroxy acid(s) and/or salt(s) thereof may be selected from citric acid, mandelic acid, glycolic acid, lactic acid, a salt thereof, and a combination of two or more thereof. In certain instances, the alpha-hydroxy acid is selected from mandelic acid, lactic acid, malic acid, tartaric acid, phytic acid, tartaric acid, alpha-hydroxy glutaric acid, gluconic acid, a salt thereof, and a combination of two or more thereof. Yet in further instances, the alpha-hydroxy acid or salt thereof is selected from lactic acid, malic acid, sodium-D-gluconate, a salt thereof, and a combination of two or more thereof. In at least one embodiment, the alpha hydroxy acid comprises lactic acid. In some preferred embodiments, alpha hydroxy acid comprises lactic acid and/or a salt thereof, mandelic acid and/or a salt thereof, and glycolic acid and/or a salt thereof.

The personal care composition typically comprises one or more beta hydroxy acid(s), preferably in an effective amount. The amount of beta hydroxy acids present in the personal care composition may be from about 0.5 to about 10 wt. %, or from about 1.5 to about 3 wt. %, relative to the total weight composition. For example, the amount of beta hydroxy acids present in the personal care composition may be from about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1 wt. %; about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; about 2 to about 10 wt. %, about 2 to about 8 wt. %, about 2 to about 6 wt. %, about 2 to about 5 wt. %, about 2 to about 4 wt. %, about 2 to about 3 wt. %; about 3 to about 10 wt. %, about 3 to about 8 wt. %, about 3 to about 6 wt. %, about 3 to about 5 wt. %, about 3 to about 4 wt. %, including ranges and subranges thereof, based on the total weight of the personal care composition. In one instance, the personal care composition comprises beta hydroxy acids from about 1.5 to about 2 wt. %, based on the total weight of the personal care composition.

The personal care composition typically comprises one or more beta hydroxy acid(s) and/or salt(s) thereof. Non-limiting examples of beta hydroxy acids include salicylic acid, propionic acid, beta-hydroybutyric acid, beta-hydroxy beta-methylbutyric acid, beta-phenyl-lactic acid, beta-phenylpyruvic acid, carnitine, a salt thereof, and combinations of two or more thereof. The beta hydroxy acids and/or salts thereof may in some cases be selected from salicylic acid, esters of salicylic acid, sodium salicylate, beta hydroxybutanoic acid, tropic acid, trethocanic acid, beta hydroxy acids obtained from white willow bark extract and/or wintergreen leaves, salt(s) thereof, and combinations of two or more thereof. In some embodiments, the beta hydroxy acid(s) comprises salicylic acid and/or sodium salicylate, beta hydroxy acids of willow bark extract, or combinations thereof. In one embodiment, the beta hydroxy acid and/or salt thereof is salicylic acid and/or sodium salicylate. In some embodiments, the beta-hydroxy acid is in salt form, e.g., a sodium salt or a potassium salt.

The personal care composition may include one or more polyhydroxy acid(s). The polyhydroxy acids may include, but are not limited to, gluconolactone, polyhydroxy benzoic acid, a polyhydroxy benzoic ester, or a combination thereof. In some embodiments, the plurality of hydroxy acids comprises a polyhydroxy benzoic acid selected from a polyhydroxy benzoic acid, a hydrate thereof, gluconolactone, and a combination of two or more thereof. The polyhydroxy benzoic acid compound may be selected from 2-hydroxy benzoic acid, 3-hydroxy benzoic acid, 4-hydroxy benzoic acid, 2, 3-dihydroxy benzoic acid, 2, 4-dihydroxy benzoic acid, 2, 5-dihydroxy benzoic acid, 2, 6-dihydroxy benzoic acid, 3, 4-dihydroxy benzoic acid, 3, 5-dihydroxy benzoic acid, 2,3, 4-trihydroxy benzoic acid, 2,4, 6-trihydroxy benzoic acid, 3,4, 5-trihydroxy benzoic acid, an ester thereof, and a combination of two or more thereof. The polyhydroxy acid may comprise gluconolactone, lactobionic acid, maltobionic acid, and a combination thereof. In at least one preferred embodiment, the one or more polyhydroxy acid comprises gluconolactone and/or a salt thereof. In some embodiments, the polyhydroxy acid is in salt form, e.g., a sodium salt or a potassium salt.

The personal care composition of the invention typically comprises polyhydroxy acid(s) in an amount from about 0.1 to about 8 wt. %, about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1 wt. %; from about 0.9 to about 8 wt. %, about 0.9 to about 6 wt. %, about 0.9 to about 5 wt. %, about 0.9 to about 4 wt. %, about 0.9 to about 3 wt. %, about 0.9 to about 2 wt. %, about 0.9 to about 1.5 wt. %; from about 1.4 to about 8 wt. %, about 1.4 to about 6 wt. %, about 1.4 to about 5 wt. %, about 1.4 to about 4 wt. %, about 1.4 to about 3 wt. %; from about 2.4 to about 8 wt. %, about 2.4 to about 6 wt. %, about 2.4 to about 5 wt. %, about 2.4 to about 4 wt. %; from about 3.4 to about 8 wt. %, about 3.4 to about 6 wt. %, about 3.4 to about 5 wt. %, about 0.5 to about 4 wt. %; from about 4.5 to about 8 wt. %, about 4.5 to about 6 wt. %, about 5.5 to about 8 wt. %, or any range or subrange thereof, based on the total weight of the personal care composition.

In some embodiments, however, the personal care composition of the invention may comprise polyhydroxy acid(s) in an amount from about 2 to about 20 wt. %, about 7 to about 20 wt. %, or from about 10% to about 15 wt. % by weight, relative to the total weight of the personal care composition. For example, the polyhydroxy acid(s) may, in some embodiments, be present in the personal care composition in an amount from about 2 to about 18 wt. %, about 2 to about 16 wt. %, about 2 to about 14 wt. %, about 2 to about 12 wt. %, about 2 to about 10 wt. %, about 2 to about 8 wt. %, about 2 to about 6 wt. %, about 2 to about 4 wt. %; from about 5 to about 20 wt. %, about 5 to about 18 wt. %, about 5 to about 16 wt. %, about 5 to about 14 wt. %, about 5 to about 12 wt. %, about 5 to about 10 wt. %, about 5 to about 8 wt. %; from about 8 to about 20 wt. %, about 8 to about 18 wt. %, about 8 to about 16 wt. %, about 8 to about 14 wt. %, about 8 to about 12 wt. %, about 8 to about 10 wt. %; from about 10 to about 20 wt. %, about 10 to about 18 wt. %, about 10 to about 16 wt. %, about 10 to about 14 wt. %, about 10 to about 12 wt. %; from about 12 to about 20 wt. %, about 12 to about 18 wt. %, about 12 to about 16 wt. %, about 12 to about 14 wt. %; from about 14 to about 20 wt. %, about 14 to about 18 wt. %, about 14 to about 16 wt. %; from about 16 to about 20 wt. %, about 16 to about 18 wt. %, including any range or subrange thereof, based on the total weight of the personal care composition. In at least one instance, the personal care composition comprises polyhydroxy acid(s) from about 10 to about 15 wt. %, based on the total weight of the personal care composition.

The personal care composition typically includes Opuntia ficus-indica (prickly pear) extract. The amount of prickly pear extract in the personal care composition may be from about 0.01 to about 5 wt. %, relative to the total weight of the personal care composition. In some embodiments, the prickly pear extract may be present in an amount from about 0.01 to about 5 wt. %, about 0.01 to about 3 wt. %, about 0.01 to about 2 wt. %, about 0.01 to about 1 wt. %, about 0.01 to about 0.75 wt. %, about 0.01 to about 0.5 wt. %, about 0.01 to about 0.25 wt. %, about 0.01 to about 0.1 wt. %; from about 0.05 to about 7 wt. %, about 0.05 to about 5 wt. %, about 0.05 to about 3 wt. %, about 0.05 to about 2 wt. %, about 0.05 to about 1 wt. %, about 0.05 to about 0.75 wt. %, about 0.05 to about 0.5 wt. %, about 0.05 to about 0.25 wt. %, about 0.05 to about 0.1 wt. %; from about 0.1 to about 7 wt. %, about 0.1 to about 5 wt. %, about 0.1 to about 3 wt. %, about 0.1 to about 2 wt. %, about 0.1 to about 1 wt. %, about 0.1 to about 0.75 wt. %, about 0.1 to about 0.5 wt. %, about 0.1 to about 0.25 wt. %; from about 0.15 to about 7 wt. %, about 0.15 to about 5 wt. %, about 0.15 to about 3 wt. %, about 0.15 to about 2 wt. %, about 0.15 to about 1 wt. %, about 0.15 to about 0.75 wt. %, about 0.15 to about 0.5 wt. %, about 0.15 to about 0.25 wt. %; from about 0.4 to about 7 wt. %, about 0.4 to about 5 wt. %, about 0.4 to about 3 wt. %, about 0.4 to about 2 wt. %, about 0.4 to about 1 wt. %, about 0.4 to about 0.75 wt. %; from about 0.8 to about 7 wt. %, about 0.8 to about 5 wt. %, about 0.8 to about 3 wt. %, about 0.8 to about 2 wt. %, about 0.8 to about 1 wt. %; from about 1.2 to about 7 wt. %, about 1.2 to about 5 wt. %, about 1.2 to about 3 wt. %, about 1.2 to about 2 wt. %; from about 1.6 to about 7 wt. %, about 1.6 to about 5 wt. %, about 1.6 to about 3 wt. %, about 1.6 to about 2 wt. %; from about 2 to about 7 wt. %, about 2 to about 5 wt. %, about 2 to about 3 wt. %, or any range or subrange thereof, based on the total weight of the personal care composition.

Additionally or alternatively, the personal care compositions may include one or more vitamin B. Examples of vitamin B include vitamin B1 (e.g., thiamine) and their derivatives, vitamin B2 (e.g., riboflavin) and their derivatives, vitamin B3 (e.g., niacin or nicotinamide/niacinamide) and their derivatives, vitamin B5 (e.g., pantothenic acid) and their derivatives, vitamin B6 (e.g., pyridoxine, pyridoxal, or pyridoxamine) and their derivatives, vitamin B12 (e.g., cyanocobalamin) and their derivatives, and combinations thereof. Vitamin B3 (e.g., niacin or nicotinamide/niacinamide) and their derivatives. Examples of Vitamin B3 include those is selected from nicotinic acid esters, non-vasodilating esters of nicotinic acid, nicotinyl amino acids, nicotinyl alcohol esters of carboxylic acids (e.g., straight-chain or branched chain, cyclic or acyclic, saturated or unsaturated (including aromatic), and substituted or unsubstituted, nicotinic acid N-oxide and niacinamide N-oxide, tocopherol nicotinate and inositol hexanicotinate. Examples of derivatives of niacinamide include nicotinyl amino acids, derived, for example, from the reaction of an activated nicotinic acid compound (e.g., nicotinic acid azide or nicotinyl chloride) with an amino acid, and nicotinyl alcohol esters of organic carboxylic acids (e.g., Cl-C18).

The personal care composition may be formulated to have vitamin B(s) in a total amount from about 0.01 to about 7 wt. %, based on the total weight of the personal care composition. In some embodiments, the total amount of vitamin B in the personal care composition may be from about 0.01 to about 5 wt. %, about 0.01 to about 3 wt. %, about 0.01 to about 2 wt. %, about 0.01 to about 1 wt. %, about 0.01 to about 0.75 wt. %, about 0.01 to about 0.5 wt. %, about 0.01 to about 0.25 wt. %, about 0.01 to about 0.1 wt. %; from about 0.05 to about 7 wt. %, about 0.05 to about 5 wt. %, about 0.05 to about 3 wt. %, about 0.05 to about 2 wt. %, about 0.05 to about 1 wt. %, about 0.05 to about 0.75 wt. %, about 0.05 to about 0.5 wt. %, about 0.05 to about 0.25 wt. %, about 0.05 to about 0.1 wt. %; from about 0.1 to about 7 wt. %, about 0.1 to about 5 wt. %, about 0.1 to about 3 wt. %, about 0.1 to about 2 wt. %, about 0.1 to about 1 wt. %, about 0.1 to about 0.75 wt. %, about 0.1 to about 0.5 wt. %, about 0.1 to about 0.25 wt. %; from about 0.15 to about 7 wt. %, about 0.15 to about 5 wt. %, about 0.15 to about 3 wt. %, about 0.15 to about 2 wt. %, about 0.15 to about 1 wt. %, about 0.15 to about 0.75 wt. %, about 0.15 to about 0.5 wt. %, about 0.15 to about 0.25 wt. %; from about 0.4 to about 7 wt. %, about 0.4 to about 5 wt. %, about 0.4 to about 3 wt. %, about 0.4 to about 2 wt. %, about 0.4 to about 1 wt. %, about 0.4 to about 0.75 wt. %; from about 0.8 to about 7 wt. %, about 0.8 to about 5 wt. %, about 0.8 to about 3 wt. %, about 0.8 to about 2 wt. %, about 0.8 to about 1 wt. %; from about 1.2 to about 7 wt. %, about 1.2 to about 5 wt. %, about 1.2 to about 3 wt. %, about 1.2 to about 2 wt. %; from about 1.6 to about 7 wt. %, about 1.6 to about 5 wt. %, about 1.6 to about 3 wt. %, about 1.6 to about 2 wt. %; from about 2 to about 7 wt. %, about 2 to about 5 wt. %, about 2 to about 3 wt. %, or any range or subrange thereof, based on the total weight of the personal care composition.

The personal care composition may include one or more skin soothing agent(s), such as aloe vera extract, allantoin, licorice extract, glycyrrhizin, or combinations thereof. In some preferable embodiments, the personal care compositions include two or more skin soothing agents. For example, the personal care composition may include two, three, four, five, six, seven, or any range thereof, of skin soothing agents. When two or more skin soothing agents are incorporated into the personal care composition, preferably at least one of the skin soothing agents is licorice extract. For example, in at least one preferable embodiment, the personal care composition includes licorice extract.

In some embodiments, the soothing agents may be selected from natural extracts comprising: Centella asiatica extract, lactobionic acid, Polygonum cuspidatum root extract, Scutellaria baicalensis root extract, Camellia sinensis leaf extract, Glycyrrhiza glabra root extract, Chamomilla recutita flower extract, Rosmarinus officinalis (rosemary) leaf extract, tea (Camellia sinensis) leaf extract, Poria cocos extract, Phragmites karka extract, lidocaine, chia seed extract, Rosa centifolia flower extract, aloe vera, allantoin, D-panthenol, turmeric, avocado oil, palmitoyl tetrapeptide-7, lichen extract, or a combination of two or more thereof.

The amount of the one or more skin soothing agent(s) in the personal care composition may be about 0.01 to about 7 wt. %, based on the total weight of the personal care composition. In some instances, the personal care composition may be formulated to have skin soothing agents(s) present in a total amount of about 0.01 to about 5 wt. %, about 0.01 to about 3 wt. %, about 0.01 to about 2 wt. %, about 0.01 to about 1 wt. %, about 0.01 to about 0.75 wt. %, about 0.01 to about 0.5 wt. %, about 0.01 to about 0.25 wt. %, about 0.01 to about 0.1 wt. %; from about 0.05 to about 7 wt. %, about 0.05 to about 5 wt. %, about 0.05 to about 3 wt. %, about 0.05 to about 2 wt. %, about 0.05 to about 1 wt. %, about 0.05 to about 0.75 wt. %, about 0.05 to about 0.5 wt. %, about 0.05 to about 0.25 wt. %, about 0.05 to about 0.1 wt. %; from about 0.1 to about 7 wt. %, about 0.1 to about 5 wt. %, about 0.1 to about 3 wt. %, about 0.1 to about 2 wt. %, about 0.1 to about 1 wt. %, about 0.1 to about 0.75 wt. %, about 0.1 to about 0.5 wt. %, about 0.1 to about 0.25 wt. %; from about 0.15 to about 7 wt. %, about 0.15 to about 5 wt. %, about 0.15 to about 3 wt. %, about 0.15 to about 2 wt. %, about 0.15 to about 1 wt. %, about 0.15 to about 0.75 wt. %, about 0.15 to about 0.5 wt. %, about 0.15 to about 0.25 wt. %; from about 0.4 to about 7 wt. %, about 0.4 to about 5 wt. %, about 0.4 to about 3 wt. %, about 0.4 to about 2 wt. %, about 0.4 to about 1 wt. %, about 0.4 to about 0.75 wt. %; from about 0.8 to about 7 wt. %, about 0.8 to about 5 wt. %, about 0.8 to about 3 wt. %, about 0.8 to about 2 wt. %, about 0.8 to about 1 wt. %; from about 1.2 to about 7 wt. %, about 1.2 to about 5 wt. %, about 1.2 to about 3 wt. %, about 1.2 to about 2 wt. %; from about 1.6 to about 7 wt. %, about 1.6 to about 5 wt. %, about 1.6 to about 3 wt. %, about 1.6 to about 2 wt. %; from about 2 to about 7 wt. %, about 2 to about 5 wt. %, about 2 to about 3 wt. %, or any range or subrange thereof, based on the total weight of the personal care composition.

The personal care composition may comprise one or more penetration agent(s). In general, penetration agents may include, but are not limited to, monoalcohols, ethanol, witch hazel, urea, fatty acids, glycols or combinations thereof. In some embodiments, the penetration agents include monoalcohols, ethanol, propanol, butanol, witch hazel, glycols or combinations thereof. For instance, the penetration agents may include monoalcohol, anhydrous ethyl alcohol, water, and witch hazel. In at least one embodiment, the penetration agents are ethanol and witch hazel.

The personal care composition may comprise one or more penetration agent(s) in an amount up to about 5 wt. %, or from about 0.5 to about 3 wt. %, relative to the total weight of the personal care composition. For instance, the amount of penetrating agent(s) present in the personal are composition may be from about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 5 wt. %, about 0.5 to about 4 wt. %, about 0.5 to about 3 wt. %, about 0.5 to about 2 wt. %, about 0.5 to about 1 wt. %; from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the personal care composition.

In general, the personal care product may include humectants including, but are not limited to, glycerin, butylene glycol, 1,3-propanediol, sorbitol, xylitol or low molecular weight polyethylene glycols (PEGs), polyoxyethylenes or combinations thereof. In some embodiments, the humectant agents are selected from sorbitol (preferably, non-crystal sorbitol), butylene glycol, 1,3-propanediol, glycerin (e.g., vegetable refined glycerin), polyoxyethylene glycol, pentylene glycol, or combinations thereof. In at least one embodiment, the humectants are chosen from glycerin, butylene glycol, and 1,3-propanediol. Typically, the humectant is vegetable refined glycerin. Other humectants include, but are not limited to, polyglutamic acid, saccharide isomerate, or combinations thereof.

The personal care composition of the invention may comprise humectants in an amount from about 0.01 to about 7 wt. %, relative to the total weight of the personal care composition. For example, the humectants may be present in the personal care composition in an amount of about 0.01 to about 5 wt. %, about 0.01 to about 3 wt. %, about 0.01 to about 2 wt. %, about 0.01 to about 1 wt. %, about 0.01 to about 0.75 wt. %, about 0.01 to about 0.5 wt. %, about 0.01 to about 0.25 wt. %, about 0.01 to about 0.1 wt. %; from about 0.05 to about 7 wt. %, about 0.05 to about 5 wt. %, about 0.05 to about 3 wt. %, about 0.05 to about 2 wt. %, about 0.05 to about 1 wt. %, about 0.05 to about 0.75 wt. %, about 0.05 to about 0.5 wt. %, about 0.05 to about 0.25 wt. %, about 0.05 to about 0.1 wt. %; from about 0.1 to about 7 wt. %, about 0.1 to about 5 wt. %, about 0.1 to about 3 wt. %, about 0.1 to about 2 wt. %, about 0.1 to about 1 wt. %, about 0.1 to about 0.75 wt. %, about 0.1 to about 0.5 wt. %, about 0.1 to about 0.25 wt. %; from about 0.15 to about 7 wt. %, about 0.15 to about 5 wt. %, about 0.15 to about 3 wt. %, about 0.15 to about 2 wt. %, about 0.15 to about 1 wt. %, about 0.15 to about 0.75 wt. %, about 0.15 to about 0.5 wt. %, about 0.15 to about 0.25 wt. %; from about 0.4 to about 7 wt. %, about 0.4 to about 5 wt. %, about 0.4 to about 3 wt. %, about 0.4 to about 2 wt. %, about 0.4 to about 1 wt. %, about 0.4 to about 0.75 wt. %; from about 0.8 to about 7 wt. %, about 0.8 to about 5 wt. %, about 0.8 to about 3 wt. %, about 0.8 to about 2 wt. %, about 0.8 to about 1 wt. %; from about 1.2 to about 7 wt. %, about 1.2 to about 5 wt. %, about 1.2 to about 3 wt. %, about 1.2 to about 2 wt. %; from about 1.6 to about 7 wt. %, about 1.6 to about 5 wt. %, about 1.6 to about 3 wt. %, about 1.6 to about 2 wt. %; from about 2 to about 7 wt. %, about 2 to about 5 wt. %, about 2 to about 3 wt. %, or any range or subrange thereof, based on the total weight of the personal care composition.

The personal care composition may include one or more carrier(s). In general, carriers include but are not limited to, water or alcohols (e.g., ethanol, isopropanol or mixtures thereof). In at least one embodiment, the carrier comprises water. The amount of carrier present in the personal care composition may vary, e.g., from about 5 to about 90 wt. %, based on the total weight of the personal care composition. In some cases, the personal care composition includes one or more carrier(s) in an amount from about 5 to about 75 wt. %, about 5 to about 50 wt. %, about 5 to about 25 wt. %, about 5 to about 15 wt. %; from about 25 to about 90 wt. %, about 25 to about 75 wt. %, about 25 to about 50 wt. %, about 25 to about 40 wt. %; from about 40 to about 90 wt. %, about 40 to about 75 wt. %, about 40 to about 60 wt. %; from about 60 to about 90 wt. %, about 60 to about 80 wt. %, about 60 to about 70 wt. %; from about 75 to about 90 wt. %, about 75 to about 85 wt. %; from about 80 to about 90 wt. %, about 85 to about 90 wt. %, or any ranges and subranges thereof, based on the total weight of the personal care composition.

In some embodiments, the amount of monoalcohol in the personal care composition may be from about 1 to about 20 wt. %, based on the total weight of the personal care composition. For example, the personal care composition may include monoalcohol(s), such as ethanol, in an amount from about 1 to about 20 wt. %, about 4 to about 20 wt. %, about 7 to about 20 wt. %, about 10 to about 20 wt. %, about 13 to about 20 wt. %; from about 1 to about 20 wt. %, about 4 to about 20 wt. %, about 7 to about 20 wt. %, about 10 to about 20 wt. %, about 13 to about 20 wt. %; from about 1 to about 17 wt. %, about 4 to about 17 wt. %, about 7 to about 17 wt. %, about 10 to about 17 wt. %, about 13 to about 17 wt. %; from about 1 to about 15 wt. %, about 4 to about 15 wt. %, about 7 to about 15 wt. %, about 10 to about 15 wt. %, about 13 to about 15 wt. %; from about 1 to about 12 wt. %, about 4 to about 12 wt. %, about 7 to about 12 wt. %, about 10 to about 12 wt. %; from about 1 to about 8 wt. %, about 4 to about 8 wt. %, about 6 to about 8 wt. %, about 1 to about 5 wt. %, or any range or subrange thereof, based on the total weight of the personal care composition.

Additionally or alternatively, the personal care composition comprises one or more antioxidant(s). Examples of antioxidants include, but are not limited to, Camellia japonica extract, quercetin, xanthophylls, chrysin, amellia japonica extract, quercetin, green tea, soy, milk thistle, algae, aloe, angelica, bitter orange, coffee, goldthread, grapefruit, hoellen, honeysuckle, Job's tears, diglucosyl gallic acid, lithospermum, mulberry, peony, puerarua, rice, safflower, and combinations thereof. Additional antioxidants include vitamin C and derivatives thereof, including ascorbic acid, sodium ascorbate, and the fat soluble esters tetrahexyldecyl ascorbate and ascorbyl palmitate, magnesium ascorbyl phosphate, ascorbyl-glucoside, glucosamine ascorbate, ascorbyl acetate, etc. In one embodiment, the antioxidant comprises Camellia Japonica extract, xanthophylls, chrysin, or a combination thereof.

The personal care composition of the invention may comprise one or more antioxidant(s) in an amount up to about 5 wt. % or, e.g., from about 0.5 to about 3 wt. %, relative to the total weight of the personal care composition. In some cases, the amount of antioxidant(s) present in the personal are composition may be from about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 5 wt. %, about 0.5 to about 4 wt. %, about 0.5 to about 3 wt. %, about 0.5 to about 2 wt. %, about 0.5 to about 1 wt. %; from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the personal care composition.

The personal care compositions of the invention may optionally comprise preservative systems, wherein the preservative systems may include, but are not limited to, imidazolidinyl urea, diazolidinyl urea, phenoxyethanol, methylparaben, ethylparaben, and propylparaben.

The personal care compositions of the invention may be formulated as one-step treatment or as multiple step treatment to be used sequentially on the skin of the human subject. The personal care compositions of the invention may optionally comprise mineral acids, wherein the mineral acids include, but are not limited to, trichloracetic acid.

The personal care compositions of the invention may optionally comprise cooling agents, wherein the cooling agents include, but are not limited to mint oil, cyclic α-keto enamines, aloe vera, chamomile, green tea, peppermint, lemon, eucalyptus and rose.

The personal care compositions of the invention may optionally comprise vitamins and vitamin derivatives, wherein the vitamins may include, but are not limited to, Vitamin A, retinyl acetate, retinyl palmitate, Vitamin C, ascorbic acid, ascorbyl palmitate, magnesium ascorbyl palmitate, magnesium ascorbyl phosphate, Vitamin E, and tocopherol.

The personal care compositions of the invention may optionally comprise botanicals, wherein the botanicals include, but are not limited to, carotenoids, polyphenols, apigenin (flavonoid occurring in numerous herbs, fruits and vegetables), quercetin (flavonol found in onion skin and apple peel), curcumin (from turmeric rhizome), silymarin (extract of flavonolignans from milk thistle), isoflavones, genistein (isoflavone from soybeans), proanthocyanidins (from seeds of grapes), and resveratrol (polyphenol found in grapes, peanuts, fruits, red wine and mulberries). The botanicals may be present in an amount from about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 5 wt. %, about 0.5 to about 4 wt. %, about 0.5 to about 3 wt. %, about 0.5 to about 2 wt. %, about 0.5 to about 1 wt. %; from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the personal care composition.

Additional or alternative humectants that may optionally be present in the personal care compositions include, but are not limited to, glucuronic acid, N-acetyl-glucosamine, hyaluronic acid of different molecular weights, different salts of hyaluronic acid (acetylated hyaluronic acid, sodium acetylated hyaluronate, sodium hyaluronate, potassium hyaluronate). For instance, the personal care composition may comprise hyaluronic acid and/or a salt thereof in an amount up to about 2 wt. %, up to about 1.5 wt. %, up to about 1 wt. %, up to about 0.5 wt. %, up to about 0.1 wt. %, up to about 0.05 wt. % up to about 0.01 wt. %; from about 0.001 to about 0.1, about 0.001 to about 0.05, about 0.001 to about 0.01; from about 0.01 to about 2 wt. %, about 0.01 to about 1.5 wt. %, about 0.01 to about 1 wt. %, about 0.01 to about 0.5 wt. %, about 0.01 to about 0.1 wt. %, about 0.01 to about 0.05 wt. %; from about 0.05 to about 2 wt. %, about 0.05 to about 1.5 wt. %, about 0.05 to about 1 wt. %, about 0.05 to about 0.5 wt. %, about 0.05 to about 0.1 wt. %; from about 0.1 to about 2 wt. %, about 0.1 to about 1.5 wt. %, about 0.1 to about 1 wt. %, about 0.1 to about 0.5 wt. %; from about 0.2 to about 2 wt. %, about 0.2 to about 1.5 wt. %, about 0.2 to about 1 wt. %, about 0.2 to about 0.5 wt. %; from about 0.5 to about 2 wt. %, about 0.5 to about 1.5 wt. %, about 0.5 to about 1 wt. %, or any range or subrange thereof, based on the total weight of the personal care composition. Additionally or alternatively, the personal care composition may comprise glucuronic acid and/or N-acetyl-glucosamine in an amount up to about 2 wt. %, up to about 1.5 wt. %, up to about 1 wt. %, up to about 0.5 wt. %, up to about 0.1 wt. %, up to about 0.05 wt. %; from about 0.01 to about 2 wt. %, about 0.01 to about 1.5 wt. %, about 0.01 to about 1 wt. %, about 0.01 to about 0.5 wt. %, about 0.01 to about 0.1 wt. %, about 0.01 to about 0.05 wt. %; from about 0.05 to about 2 wt. %, about 0.05 to about 1.5 wt. %, about 0.05 to about 1 wt. %, about 0.05 to about 0.5 wt. %, about 0.05 to about 0.1 wt. %; from about 0.1 to about 2 wt. %, about 0.1 to about 1.5 wt. %, about 0.1 to about 1 wt. %, about 0.1 to about 0.5 wt. %; from about 0.2 to about 2 wt. %, about 0.2 to about 1.5 wt. %, about 0.2 to about 1 wt. %, about 0.2 to about 0.5 wt. %; from about 0.5 to about 2 wt. %, about 0.5 to about 1.5 wt. %, about 0.5 to about 1 wt. %, or any range or subrange thereof, based on the total weight of the personal care composition.

The personal care compositions of the invention may optionally comprise antimicrobials, wherein the antimicrobials may include, but are not limited to, benzoyl peroxide, erythromycin, tetracycline, triclosan, azelaic acid, clindamycin, chlorhexidine, tetracycline, neomycin, miconazole, clotrimazole.

The personal care compositions of the invention may optionally comprise aesthetic components such as fragrances, pigments, colorants, essential oils, skin sensates, and astringents. Such aesthetic components include, but are not limited to, clove oil, menthol, camphor, eucalyptus oil, eugenol, menthyl lactate, bisabolol, green tea extract, and lecithin. The aesthetic components may be present in an amount from about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 5 wt. %, about 0.5 to about 4 wt. %, about 0.5 to about 3 wt. %, about 0.5 to about 2 wt. %, about 0.5 to about 1 wt. %; from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the personal care composition.

The personal care compositions of the invention may optionally include or, in some cases exclude, antimicrobial agents, binders skin bleaching agents, and thickening agents. The personal care compositions of the invention may optionally comprise skin bleaching agents, wherein the skin bleaching agents include, but are not limited to, hydroquinone, kojic acid, sodium metabisulfite, licorice extract, resorcinol, phenylethyl resorcinol. The amount of bleaching agents, if present, in the personal care composition may be from about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 5 wt. %, about 0.5 to about 4 wt. %, about 0.5 to about 3 wt. %, about 0.5 to about 2 wt. %, about 0.5 to about 1 wt. %; from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the personal care composition.

The personal care product may comprise a pad or applicator. The pad may comprise a single layer or multiple layers. The personal care composition may be applied to the pad using any means currently known in the art including, but not limited to, impregnation, soaking, layering, or injection. The personal composition may be applied to the whole pad or a portion of the pad such as a single layer in a multi-layer pad. In some embodiments, the personal care composition may be a gel layer deposited on the pad or between the layers of the pad. The pad may be made from any known material in the art including, but not limited to, natural or synthetic fibers, natural porous materials, cotton or a cotton/synthetic blend, silk, wool, bamboo, artificial fiber matrix cellulosic fibers, acrylics, polyester, nylon, or combination thereof.

In some embodiments, the pad may comprise one or more abrasive elements. The one or more abrasive elements may include a plurality of protruding members, an abrasive surface, a foam pad, another type of pad, and/or the like. In some embodiments, the abrasive element may be in the personal care composition or may be a separate composition.

In one embodiment, the present invention provides personal care products and compositions useful in exfoliating the skin, increasing natural skin exfoliation, brightening the skin, smoothing skin texture, toning the skin, promoting even skin tone, encouraging cell turnover in the skin, preventing clogged pores, promoting clear complexion, reducing fine wrinkles and lines, reducing pore size, exfoliating the skin, eliminating acne, toning the skin, enhancing the skin's radiance, and providing softer, smoother skin with a more uniform appearance.

In one embodiment, the present invention provides a method to exfoliate and/or brighten the skin by applying a personal care composition to the skin with an exfoliation pad.

In one embodiment, the present invention provides a method to increase the skin's cell turnover rate by stimulating the epidermis to produce fresh skin, bringing new skin cells to the surface, reducing signs of fine lines, wrinkles, dark spots and clearing breakouts by treating the skin with a personal care product comprising a pad comprising a personal care composition.

### EXAMPLES

The following examples are for illustrative purposes only and are not intended, nor should they be construed as limiting the invention in any manner. Those skilled in the art will appreciate that variations and modifications of the following examples may be made without exceeding the spirit or scope of the invention.

### Example 1

Two non-limiting, example personal care composition (Example Compositions A and B) were prepared in accordance with aspects of the invention. The formulation for Example Compositions A and B are shown in Tables 1 and 2, below.

TABLE 1

| Ingredient | Example A (wt. %) |
|---|---|
| Hydroxy Acid | 8-10 |
| Hydrolyzed Opuntia Ficus | 0.01-0.3 |
| Indica Flower (prickly pear | |
| cactus) Extract | |
| Niacinamide | 2-5 |
| Diglucosyl Gallic Acid | 0.01-0.3 |
| Xanthophylls and Chrysin | 0.05-0.6 |
| Lecithin | 0 < x ≤ 0.1 |
| Rosmarinus Officinalis | 0 < x ≤ 0.1 |
| (Rosemary) Leaf Extract | |
| Palmitoyl Tetrapeptide-7 | 0 < x ≤ 0.1 |
| Caprylyl glycol, | 0 < x ≤ 1 |
| Phenoxyethanol, Sodium | |
| ascorbate, and Tocopherol | |
| Glycerin and Pentylene Glycol | 1-3 |
| Licorice Extract | 0.02-0.03 |
| Ethanol | 10.2 |
| Water | Q.S. to 100 |

TABLE 2

| Ingredient | Example B (wt. %) |
|---|---|
| Lactic Acid | 4.1 |
| Glycolic Acid | 3 |
| Salicylic Acid | 0.5 |
| Mandelic Acid | 0.23 |
| Gluconolactone | 0.23 |
| Maltobionic acid | 0.2 |
| Glycerin | 3.5 |
| Pentylene Glycol | 5.2 |
| Niacinamide | 2 |
| Hydrolyzed Opuntia Ficus | 0.01-0.1 |
| Indica Flower (prickly pear | |
| cactus) Extract | |
| Rosmarinus Officinalis | 0.001-0.1 |
| (Rosemary) Leaf Extract | |
| Sodium Hyaluronate | 0.01-0.05 |
| Resveratrol | 0.05-0.15 |
| Ricinoleth-40 | 0.2-0.6 |
| Palmitoyl Tetrapeptide-7 | 0.001-0.01 |
| Chrysin | 0.0001-0.01 |
| Diglucosyl Gallic Acid | 0.01-0.05 |
| Lactobionic acid | 0.2-0.3 |
| Licorice Extract | 0.02-0.03 |
| Yeast Extract | 0.01-0.05 |
| Preservatives (Caprylyl glycol, | 0.9-1.5 |
| 1,2-hexanediol, | |
| Ethylhexylglycerin, | |
| Phenoxyethanol, Potassium | |
| Sorbate, and Sodium Benzoate) | |
| Sodium Hydroxide | 0.8-1.4 |
| Ethanol | 10.2 |
| Water | Q.S to 100 |

### Example 2

Ex vivo skin evaluations were performed to assess the effect of Example Composition B on IL-la cytokine expression, ELOVL4 gene expression, KLK7 gene expression, KRT 5 gene expression, and filaggrin protein expression. Specifically, the ex vivo human skin model employed human skin explant samples from a 45-year-old female with skin having a Fitzpatrick 2 coloration. The tissues were topically treated once with Example Composition B in quadruplicate, and tissue incubation continued for 6 days. A quadruplicate set of tissues were cultured for the same length of time, but left untreated with any composition as a control. RNA was isolated from these tissues, and assessed for changes in the gene expression of ELOVL4, key to barrier lipid synthesis and epidermal barrier formation, and KLK7, a key enzyme for desquamation.

Separately, a reconstructed epidermal model, was treated topically with Example Composition B, and allowed to incubate for 24 hours post treatment. The treated samples were in quadruplicate, with another quadruplicate untreated sample serving as control. Any IL-la cytokine secreted by the tissues within 24 hours post treatment was evaluated using ELISA. Filaggrin protein expression in these tissues were also evaluated by ELISA, and relative KRT5 expression was quantified using qPCR.

Two comparative compositions (Comparative Compositions 1 and 2) were evaluated according to the same procedure as Example Composition B to serve as a comparison for the level of IL-la cytokine expression. The list of ingredients for Comparative Composition 1 was: water, sucrose, Cucurbita pepo, propylene glycol, lactic acid, citric acid, octoxynol-9, fragrance, gluconic acid, yeast, aminoguanidine HCL, cinnamomum cassia leaf oil, Eugenia caryophyllus leaf oil, glutathione, Zingiber officinale root oil, alcohol denat., zinc salicylate, aspergillus/molasses ferment filtrate, glycerin, potassium chloride, benzyl benzoate, eugenol, coumarin, cinnamal, isoeugenol, and cinnamyl alcohol. The list of ingredients for Comparative composition 2 was: water, lactic acid, citric acid, glycerin, Arctium lappa root extract, Calendula officinalis flower extract, Cucumis sativus fruit extract, Hedera helix leaf/stem extract, Hydrastis canadensis extract, Paullinia cupana seed extract, Salvia officinalis leaf extract, tetrasodium EDTA, and aloe barbadensis leaf juice.

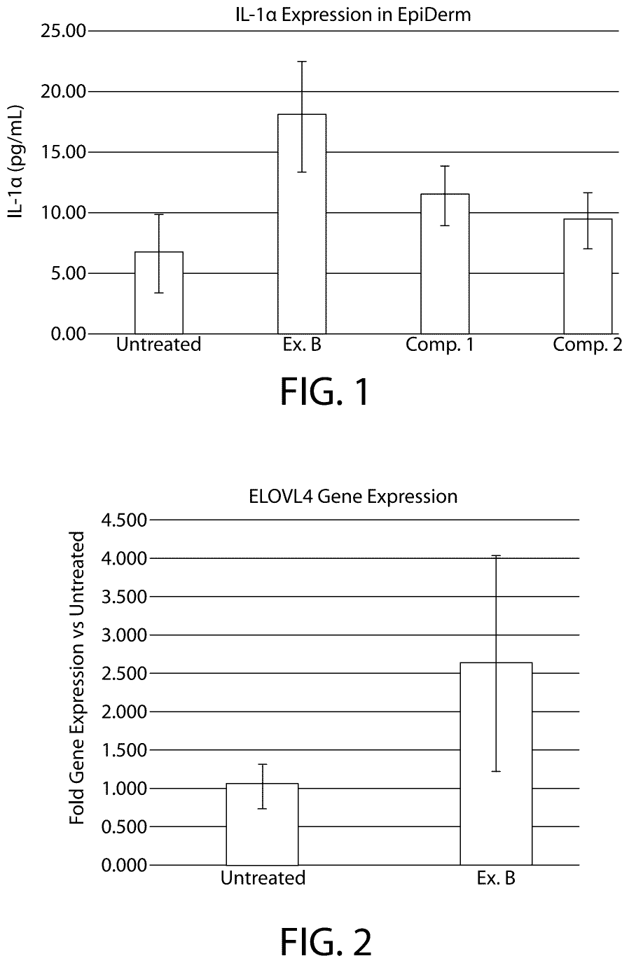

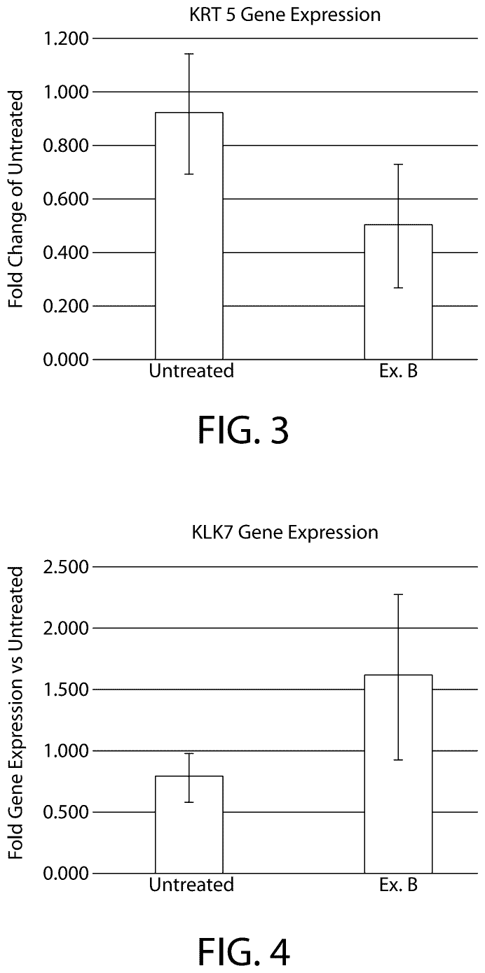

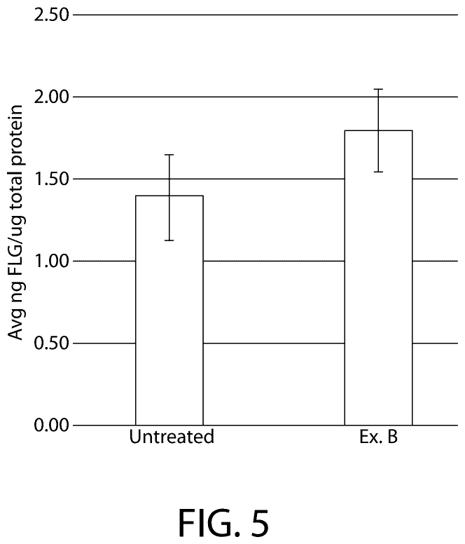

The results for evaluation of Example Composition B on IL-la cytokine expression, ELOVL4 gene expression, KLK7 gene expression, KRT 5 gene expression, and filaggrin protein expression are shown in FIGS. 1-5.

### Example 3

Two non-limiting, example personal care compositions (Example Compositions C and D) were prepared in accordance with aspects of the invention. The formulation for Example Compositions C and D are shown in Tables 3 and 4, below.

TABLE 3

| Ingredient | Example C (wt. %) |
|---|---|
| Hydroxy Acid | 8-10 |
| Hydrolyzed Opuntia Ficus | 0.01-0.3 |
| Indica Flower (prickly pear | |
| cactus) Extract | |
| Niacinamide | 2-5 |
| Chrysin | 0.0001 < x < 0.01 |
| Rosmarinus Officinalis | 0 < x ≤ 0.1 |
| (Rosemary) Leaf Extract | |
| Palmitoyl Tetrapeptide-7 | 0 < x ≤ 0.1 |
| Caprylyl glycol, | 0 < x ≤ 1 |
| Phenoxyethanol, Sodium | |
| ascorbate, and Tocopherol | |
| Glycerin and Pentylene Glycol | 1-3 |
| Licorice Extract | 0.02-0.03 |
| Ethanol | 10.2 |
| Water | Q.S. to 100 |

TABLE 4

| Ingredient | Example D (wt. %) |
|---|---|
| Lactic Acid | 8 |
| Glycolic Acid | 4 |
| Salicylic Acid | 0.5 |
| Mandelic Acid | 0.3 |
| Gluconolactone | 0.3 |
| Maltobionic acid | 0.3 |
| Lactobionic acid | 0.2-0.3 |
| Glycerin | 2.3 |
| Pentylene Glycol | 5.1 |
| Niacinamide | 2 |
| Hydrolyzed Opuntia Ficus | 0.01-0.1 |
| Indica Flower (prickly pear | |
| cactus) Extract | |
| Rosmarinus Officinalis | 0.001-0.1 |
| (Rosemary) Leaf Extract | |
| Sodium Hyaluronate | 0.001-0.05 |
| Resveratrol | 0.05-0.15 |
| Palmitoyl Tetrapeptide-7 | 0.001-0.01 |
| Chrysin | 0.0001-0.01 |
| Licorice Extract | 0.02-0.03 |
| Yeast Extract | 0.01-0.05 |
| Preservatives (Caprylyl Glycol, | 0.9-1.5 |
| 1,2-Hexanediol, | |
| Ethylhexylglycerin, | |
| Phenoxyethanol, Potassium | |
| Sorbate, t-Butyl Alcohol, | |
| Denatonium Benzoate, | |
| Tocopherol, and Sodium | |
| Benzoate) | |
| Sodium Hydroxide | 2.3 |
| Ethanol | 10.2 |
| Water | Q.S to 100 |

What is claimed is:

1. A personal care product comprising:
a pad containing a personal care composition, the personal care composition comprising:
from about 2 to about 23 wt. % of a plurality of hydroxy acids;
from about 0.01 to about 7 wt. % of prickly pear; and
from about 1 to about 12 wt. % of vitamin B.

2. The personal care product according to claim 1, wherein the plurality of hydroxy acids comprise an alpha hydroxy acid and a beta hydroxy acid.

3. The personal care product according to claim 1, wherein the plurality of hydroxy acids comprise an alpha hydroxy acid selected from the group consisting of glycolic acid, citric acid, lactic acid, mandelic acid, malic acid, tartaric acid, phytic acid, hydroxycaprylic acid, hydroxycapric acid, and a combination of two or more thereof.

4. The personal care product according to claim 2, wherein the alpha hydroxy acid comprises mandelic acid, lactic acid, malic acid, gluconic acid, tartaric acid, phytic acid, or a combination of two or more thereof.

5. The personal care product according to claim 2, wherein the alpha hydroxy acid is present in an amount from 1 to about 16 wt. %, based on the total weight of the personal care composition.

6. The personal care product according to claim 1, wherein the plurality of hydroxy acids comprise a beta hydroxy acid selected from the group consisting of salicylic acid, sodium salicylate, beta hydroxybutanoic acid, tropic acid, trethocanic acid, and a combination of two or more thereof.

7. The personal care product according to claim 2, wherein the beta hydroxy acid comprises salicylic acid, sodium salicylate, or a combination thereof.

8. The personal care composition according to claim 2, wherein the beta hydroxy acid is present in an amount from about 0.5 to about 10 wt. %, based on the total weight of the personal care composition.

9. The personal care product according to claim 1, wherein the plurality of hydroxy acids comprise a polyhydroxy benzoic acid, a polyhydroxy benzoic ester, a hydrate thereof, or a combination thereof.

10. The personal care product according to claim 9, wherein the polyhydroxy benzoic acid compound is selected from the group consisting of gluconolactone, 2-hydroxy benzoic acid, 3-hydroxy benzoic acid, 4-hydroxy benzoic acid, 2, 3-dihydroxy benzoic acid, 2, 4-dihydroxy benzoic acid, 2, 5-dihydroxy benzoic acid, 2, 6-dihydroxy benzoic acid, 3, 4-dihydroxy benzoic acid, 3, 5-dihydroxy benzoic acid, 2,3, 4-trihydroxy benzoic acid, 2,4, 6-trihydroxy benzoic acid, 3,4, 5-trihydroxy benzoic acid, an ester thereof, and a combination of two or more thereof.

11. The personal care product according to claim 1, further comprising a soothing agent, optionally, wherein the soothing agent is present in an amount from about 0.01 to about 7 wt. %, based on the total weight of the personal care composition.

12. The personal care product according to claim 11, wherein the soothing agent comprises Centella asiatica extract, Polygonum cuspidatum root extract, Scutellaria baicalensis root extract, Camellia sinensis leaf extract, Glycyrrhiza glabra (licorice) root extract, Chamomilla recutita (matricaria) flower extract, Rosmarinus officinalis (rosemary) leaf extract, tea (Camellia sinensis) leaf extract, Poria cocos extract, Phragmites karka extract, lidocaine, chia seed extract, Rosa centifolia flower extract, aloe vera, allantoin, D-panthenol, turmeric, avocado oil, and lichen extract, or a combination of two or more thereof.

13. The personal care product according to claim 1, further comprising a humectant, optionally, in an amount from about 1 to about 20 wt. %, based on the total weight of the personal care composition.

14. The personal care product according to claim 13, wherein the humectant is selected from the group consisting of glycerin, butylene glycol, 1,3-propanediol, polyglutamic acid, saccharide isomerate, and a combination of two or more thereof.

15. The personal care composition according to claim 1, wherein the vitamin B is selected from the group consisting of vitamin B1, vitamin B2, vitamin B3, vitamin B5, vitamin B6, vitamin B12, a derivative thereof, and a combination of two or more thereof.

16. The personal care composition according to claim 1, wherein the vitamin B is selected from the group consisting of thiamine, riboflavin, niacinamide, pantothenic acid, and a combination of two or more thereof.

17. The personal care composition according to claim 1, wherein the plurality of hydroxy acids comprises two or more alpha hydroxy acids selected from the group consisting of lactic acid, glycolic acid, mandelic acid, gluconic acid, and/or salts thereof.

18. A personal care product comprising:
a pad; and
a personal care composition according to any foregoing claim, wherein the personal care composition is contained in the pad.

19. The personal care product according to claim 18, wherein the personal care composition is impregnated in the pad.

20. The personal care product according to claim 18, wherein the pad comprises a gel phase.
